(19) United States
(12) Patent Application Publication
(10) Pub. No.: US 2025/0186435 A1
(43) Pub. Date: Jun. 12, 2025

(54) PHARMACEUTICAL FORMULATIONS AND THEIR PREPARATIONS FOR TREATMENT OF CANCER

(71) Applicant: Cellix Bio Private Limited, Hyderabad (IN)

(72) Inventor: Mahesh Kandula, East Godavari District, Andhra Pradesh (IN)

(73) Assignee: Cellix Bio Private Limited, Hyderabad (IN)

(21) Appl. No.: 17/926,992

(22) PCT Filed: May 17, 2021
(86) PCT No.: PCT/IB2021/054199

§ 371 Date: Aug. 28, 2023

(30) Foreign Application Priority Data
May 19, 2020 (IN) 202041020998

(51) Int. Cl. A61K 31/506; A61K 9/20

(52) U.S. Cl. CPC A61K 31/506; A61K 9/2013; A61K 9/2018; A61K 9/2027; A61K 9/2054

(57) ABSTRACT

The present invention relates to a pharmaceutical formulation of 5-fluoropyrimidine derivatives or a salt, solvate, or hydrate thereof for oral administration wherein the formulation is a simple composition for ingestion, simple, safest, convenient, non-invasive, versatility and most importantly, the patient compliance for oral administration with improved compliance, safety, and bioavailability. Further disclosed is a process for the preparation, composition, methods of administration, dosages of the formulation and their use for treating cancers and its associated complications.

### FIELD OF THE INVENTION

The present invention relates to a pharmaceutical formulation of 5-fluoropyrimidine derivatives or a salt, solvate, or hydrate thereof further the present invention discloses process for the preparation, composition, methods of administration, dosages of the said formulation.

### BACKGROUND OF THE INVENTION

Pyrimidine analogues are nucleoside analog antimetabolites which mimic the structure of metabolic pyrimidines and the examples includes 5-Fluorouracil (5-FU), Floxuridine (FUDR), Cytarabine (Cytosine arabinoside), 6-azauracil (6-AU), gemcitabine which inhibits thymidylate synthase.

Fluoropyrimidine carbamate is a class of major anticancer drugs also referred as 5-fluorouracil (5-FU) prodrug, capecitabine is used in treating solid tumors such as breast, gastrointestinal, head and neck and colorectal cancers. Capecitabine (XELODA® by Roche) is an orally administered prodrug of 5′-deoxy-5-fluorouridine (5′-DFUR) which is converted to 5-fluorouracil.

The oral fluoropyrimidines may be divided into three groups: (a) 5-FU prodrugs; (b) 5-FU combined with a dihydropyrimidine dehydrogenase (DPD) inhibitor, and (c) 5-FU prodrugs combined with a dihydropyrimidine dehydrogenase (DPD) inhibitor.

Currently available immediate release (IR) orally administered anticancer fluoropyrimidine drug(s) are unable to maintain the plasma concentration of the drug within the desired therapeutic effective range. After oral administration of currently available capecitabine IR composition of anticancer drug the plasma concentration of the drug reaches below minimum therapeutic effective concentration approximately after 6 hours, which results in suboptimal therapeutic effect.

5-fluoropyrimidine derivatives is an example of a prodrug of 5-FU with a pharmacological mode of action like capecitabine. Endogenous enzymes convert 5-fluoropyrimidine derivatives to 5-fluorouracil (5-FU). Both normal and tumor cells metabolize 5-FU to 5-fluoro-2′-deoxyuridine monophosphate (FdUMP) and 5-fluorouridine triphosphate (FUTP).

Hence, there is a need to develop anticancer composition/formulation which could overcome the shortcomings associated with currently available capecitabine IR tablet(s), and modified releases dosage forms of the anticancer drug.

### OBJECTS OF THE INVENTION

Present invention provides a pharmaceutical formulation of 5-fluoropyrimidine derivatives, wherein the composition comprises immediate release or controlled release or delayed release or sustained release formulations and dosage forms comprising of 5-fluoropyrimidine derivatives, for an oral administration. Further the invention provides process for the preparation of said composition.

In an embodiment, present invention provides a pharmaceutical formulation of 5-fluoropyrimidine derivatives, wherein the formulation is easy for ingestion, simple, safe, convenient, non-invasive, and most importantly, the patient compliance for oral administration with improved compliance solubility, stability and bioavailability.

In another embodiment present invention provides solid/semi-solid/liquid dosages forms for oral administration which includes pills, tablets, capsules, lozenges/troches, pastilles, powders and granules.

In certain embodiments, the present invention also provides pharmaceutical formulation of 5-fluoropyrimidine derivatives administered through parenteral routes such as not limited to injection, intravenous, intramuscular, pump-based infusion, depot release as well as topical formulations.

In some embodiment of the present invention provide a tablet dosage formulation that are intended to be swallowed whole, disintegrate, and release their medicaments quickly/in regulated manner after oral administration.

In certain embodiment, the present invention provides a pharmaceutical formulation for immediate/rapidly release of 5-fluoropyrimidine derivatives with decreased dissolution and disintegration times for immediate release by using suitable diluents and super disintegrants with no special rate controlling features.

In an embodiment present invention provide a pharmaceutical formulation with ideal dosage regimen of drug therapy for immediate release and the desired therapeutic concentration of drug in plasma (or at the site of action) and maintains it constantly for the entire duration treatment.

In another embodiment present invention provide a pharmaceutical formulation with ideal dosage regiment of drug therapy for modified or controlled or sustained or prolonged or extended or delayed release of drug.

In an embodiments present invention provide, pharmaceutical formulation comprising a therapeutically effective amounts of 5-fluoropyrimidine derivatives. Specific embodiments herein provide, inter alia, pharmaceutical formulation comprising a therapeutically effective amounts of 5-fluoropyrimidine derivatives, wherein the formulation releases the 5-fluoropyrimidine derivatives, substantially/controlled form in the gastrointestinal tract following oral administration to a subject. The dosage form can be with enteric coating.

Further embodiments provide inter alia, pharmaceutical formulation comprising a therapeutically effective amounts of 5-fluoropyrimidine derivatives, for immediate release without enteric coating.

In one embodiment 5-fluoropyrimidine derivatives, is present in an amount of about 100-3000 mg per tablet/any dosage form. Further each dosage form may have about 150 mg, 250 mg, 300 mg, 350 mg, 450 mg, 500 mg, 650 mg, 750 mg, 850 mg, 950 mg or 1000 mg.

In an embodiment present invention provides a pharmaceutical formulation in the form of tablet or other dosage forms designed for repetitive, intermittent dosing from one or more immediate release units incorporated into a single dosage form.

In an embodiment present invention provide a pharmaceutical formulation in the form of tablet or other dosage form designed for repetitive, intermittent dosing from one or more modified/controlled release units incorporated into a single dosage form.

In an embodiment present invention provides method of manufacturing/preparing the pharmaceutical formulation comprising 5-fluoropyrimidine derivatives, wherein, the common preparation methods include molding, lyophilization or freeze drying, direct compression, spray drying, sublimation and thereof.

In an embodiment pharmaceutically acceptable composition comprising: an active ingredient selected from 5-fluropyrimidine derivatives, their isomers, stereoisomers, diastereomers, enantiomers, prodrug and pharmaceutically acceptable salts thereof in an amount of 100-3000 mg and comprising at least 60-85% by weight of the formulation: (b) one or more disintegrants or super disintegrants in an amount of 0.5 to 6% by weight; (c) one or more surfactants in an amount of 0.1 to 5% by weight; and (d) one or more binders in an amount of 0.2 to 5% by weight; and (e) at least one lubricant in an amount of 0.3 to 3% by weight; wherein the pharmaceutically acceptable composition exhibits a friability of not more than 2.5%.

In an embodiment, pharmaceutically acceptable composition comprises at least 500 mg 5-fluropyrimidine; derivative wherein 5-fluropyrimidine is derivative is (2R, 3R, 4R, 5R)-2-(5-Fluoro-4-octanamido2-oxopyrimidine-1 (2H)-yl)-5-methyl-tetrahydrofuran-3,4-diyl-diacetate.

In an embodiment pharmaceutically acceptable composition comprises one or more disintegrants or super disintegrants which are selected from polyvinylpyrrolidone, croscarmellose sodium, crospovidone, sodium starch glycolate, sodium carboxymethyl cellulose, calcium carboxymethyl cellulose, methyl cellulose, microcrystalline cellulose, powdered cellulose, lower alkyl-substituted hydroxypropyl cellulose, polacrilin potassium, starch, pregelatinized starch and sodium alginate.

In an embodiment, pharmaceutically acceptable composition comprises one or more binders which are selected from acacia, carbomer, carboxymethylcellulose, cellulose microcrystalline, croopovidone, gelatin, glucose, lactose, guar gum, hydroxypropyl cellulose, low-substituted hydroxypropyl cellulose, hydroxypropyl methylcellulose, hydroxypropyl methylcellulose, ethyl cellulose, acetate succinate, methyl cellulose, ethyl cellulose, polyethylene oxide, sodium alginate, povidone, starch, pregelatinized starch, ammonia methacrylate copolymer.

In an embodiment, pharmaceutically acceptable composition comprises one or more surfactant which are selected from sodium lauryl sulfate, ammonium lauryl sulfate, dioctyl sodium sulfosuccinate, perfluorooctanesulfonate, Lauryldimethylamine oxide, Tween80, and bile salts.

In an embodiment, pharmaceutically acceptable composition comprises at least one lubricant which are selected from calcium stearate, glyceryl behenate, magnesium stearate, mineral oil light, polyethylene glycol, castor oil, sodium stearyl fumarate, starch, stearic acid, talc, paraffin, hydrogenated vegetable oil, zinc stearate, sodium lauryl sulphate, sodium benzoate, PEG 400 and PEG 600.

In an embodiment pharmaceutically acceptable composition is a modified release composition such as sustained release, prolonged release, delayed release, pulsed release, controlled release, accelerated release, fast release, targeted release, gastric retention dosage release and thereof.

In an embodiment the pharmaceutically acceptable composition as is administered for treating cancer.

In an embodiment present invention provide methods for using 5-fluoropyrimidine derivatives, to treat, prevent, or manage diseases and disorders related to abnormal cell proliferation such as cancer more specifically solid tumor. In one embodiment, the cancer is relapsed or refractory which includes cancer of breast, lung, head and neck, ovary, testicle, prostate, gastrointestinal system, stomach, pancreas, liver, colon, kidney, bladder, brain, skin, or bone, among others. In particular embodiments, the cancer of the bladder, ovary, pancreas, lung, colon, head and neck, or breast.

### DETAILED DESCRIPTION OF THE INVENTION

As used in the specification and the accompanying claims, the indefinite articles “a” and “an” and the definite article “the” include plural as well as singular referents, unless the context clearly dictates otherwise.

As used herein, and unless otherwise specified, the terms “therapeutically effective amount” and “effective amount” of a compound/drug mean an amount sufficient to provide a therapeutic benefit in the treatment or management of a disease or disorder, or to delay or minimize one or more symptoms associated with the disease or disorder. A “therapeutically effective amount” and “effective amount” of a compound/drug mean an amount of therapeutic agent, alone or in combination with one or more other agent(s), which provides a therapeutic benefit in the treatment or management of the disease or disorder. The terms “therapeutically effective amount” and “effective amount” can encompass an amount that improves overall therapy, reduces or avoids symptoms or causes of disease or disorder, or enhances the therapeutic efficacy of another therapeutic agent.

As used herein, the terms “cancer” and “cancerous” refer to or describe the physiological condition in mammals that is typically characterized by uncontrolled cell growth. Examples of cancer include, but are not limited to lymphoma, leukemia and solid tumors. The term “Tumor,” as used herein, refers to all neoplastic cell growth and proliferation, whether malignant or benign, and all pre-cancerous and cancerous cells and tissues.

As used herein, and unless otherwise specified, the term “anticancer agent” or “cancer therapeutic agent” or “anti-cancer drug” is meant to include anti-proliferative agents and chemotherapeutic agents belonging of class of pyrimidine analogues.

The term “5-fluoropyrimidine derivatives” includes any and all possible isomer, stereoisomer, enantiomer, diastereomer, tautomer, pharmaceutically acceptable salts, hydrate, solvates, and prodrugs of the 5-fluoropyrimidine derivatives described herein.

In certain embodiments, disclosed herein encompass the use of 5-fluoropyrimidine derivatives for the preparation of a pharmaceutical formulation for treating a condition associated with abnormal cell proliferation, wherein the composition is prepared for oral administration.

As used herein, and unless otherwise specified, the term “pharmaceutically acceptable carrier or excipient or diluent,” “physiologically acceptable carrier or excipient or diluent,” refers to a pharmaceutically acceptable inert material or ingredients or vehicle, such as, e.g., a liquid or solid filler, diluent, excipient, solvent, thereof. In one embodiment, each component is “pharmaceutically acceptable” in the sense of being compatible with the other ingredients of a pharmaceutical formulation, and suitable for use in humans and animals without causing toxicity, irritation, allergic response, immunogenicity, or complications. See, e.g., Remington, The Science and Practice of Pharmacy, 21 st Edition; Lippincott Williams & Wilkins: Philadelphia, Pa., 2005; Handbook of Pharmaceutical Excipients, 5th Edition; Rowe et al., ed., The Pharmaceutical Press and the American Pharmaceutical Association: 2005; and Handbook of Pharmaceutical Additives, 3rd Edition; Ash and Ash ed., Gower Publishing Company: 2007; Pharmaceutical Preformulation and Formulation, Gibson ed., CRC Press LLC: Boca Raton, Fla., 2004.

Present invention provides a pharmaceutical formulation of 5-fluoropyrimidine derivatives, or a salt, solvate, or hydrate thereof for oral administration and release within the patients' body. The pharmaceutical formulation typically contains a therapeutically effective amount of the pharmaceutical active ingredient along with one or more inert excipient/carrier materials.

The present invention further discloses a pharmaceutical formulation of 5-fluoropyrimidine derivatives, or a salt, solvate, or hydrate thereof, wherein the formulation is easy for ingestion, simple, safest, convenient, non-invasive, and most importantly, the patient compliance for oral administration with improved compliance solubility, stability, and bioavailability.

In certain embodiments, the present invention also provides pharmaceutical formulation of 5-fluoropyrimidine derivatives administered through parenteral routes such as not limited to injection, intravenous, intramuscular, pump-based infusion, depot release as well as topical formulations.

In some embodiments, present invention provides a pharmaceutical formulation, comprises a 5-fluoropyrimidine derivatives, as an active ingredient, in combination with one or more pharmaceutically acceptable inert excipient or carrier. In one embodiment, the pharmaceutical formulation comprises at least one release controlling and at least one non-release controlling excipient or carrier.

In certain embodiments, the 5-fluoropyrimidine derivatives are used in the pharmaceutical formulations in a solid dosage form. Suitable solid forms include but are not limited to the free base of the 5-fluoropyrimidine derivatives, and solid forms comprising various salts of the 5-fluoropyrimidine derivatives. In certain embodiments, solid forms provided herein include polymorphs, solvates (including hydrates), and cocrystals comprising the 5-fluoropyrimidine derivatives and/or salts thereof. In certain embodiments, the solid form is a crystal form of the 5-fluoropyrimidine derivatives, or a pharmaceutically acceptable salt, solvate, or hydrate thereof.

In one embodiment, the pharmaceutical formulation provided herein may be formulated in various dosage forms for oral administration. The pharmaceutical formulation may also be formulated as immediate release form; or regulated release dosage forms, including modified, extended, sustained, prolonged, delayed, pulsed, controlled, accelerated, rapidly/fast, targeted, gastric retention dosage forms and thereof. These formulations are prepared according to known methods and techniques (For Ref see: Remington, The Science and Practice of Pharmacy, 21st Edition: Lippincott Williams & Wilkins: Philadelphia, Pa., 2005: Modified-Release Drug Delivery Technology, Rathbone et al., eds., Drugs and the Pharmaceutical Science, Marcel Dekker, Inc.: New York, N.Y., 2003; Vol. 126).

In some embodiments, the formulation is an immediate release tablet (IR). In some embodiments, the formulation is a controlled release tablet. In some embodiments, the formulation is a hard gelatin capsule. In certain embodiments, the formulation is a soft gelatin capsule. In certain embodiments, the formulation is an immediate release capsule. In some embodiments, the formulation is a controlled release capsule.

In certain embodiment, the pharmaceutical formulation disclosed herein may be prepared in a unit-dosage form or multiple-dosage form.

In some embodiment, the frequency of administration of pharmaceutical formulation may be administered once or multiple times, at predetermined intervals of time. It is understood in the art that the precise dosage and duration of treatment may vary with the age, weight, and condition of the patient being treated, and may be determined using known protocols from in vivo or in vitro test. It is further understood that for any particular individual, specific dosage regimens should be adjusted over time according to the individual need and the professional judgment of the physician administering or supervising the administration of the formulations.

Provided herein are dosage forms, pharmaceutical formulations, comprising 5-fluoropyrimidine derivatives that delivers 5′-DFCR into the Gastrointestinal (GI) and systemic circulation upon oral administration. In certain embodiments, the 5-fluoropyrimidine derivatives is, for example: (2R, 3R, 4R, 5R)-2-(5-Fluoro-4-octanamido 2-oxopyrimidine-1 (2H)-yl)-5-methyltetrahydrofuran-3,4-diyldiacetate or any of the compound that are disclosed in WO2018/002739 along with the method of synthesis or 5-fluoropyrimidine derivatives provided herein may be prepared using previously disclosed synthetic procedures available to a person of ordinary skill in the art. WO2018/002739 is incorporated herein by reference in their entireties.

The present invention provides solid/semi-solid/liquid dosage forms for oral administration. Suitable oral dosage forms include, but not limited to pills, tablets, capsules, lozenges/troches, pastilles, powders, powders and/or granules, sachets, emulsion, solutions, suspension, syrups, and thereof. In addition to the active ingredient, the formulation may further contain one or more pharmaceutically acceptable carriers or excipients or diluent, including, but not limited to, binders, fillers, diluents, disintegrants, surfactants, pH modifier, wetting agents, lubricants, permeation enhancer, glidants, coloring agents, dye-migration inhibitors, and flavoring agents.

According to present invention, binder may include, but not limited to acacia, carbomer, carboxymethylcellulose, cellulose microcrystalline, copovidone, gelatin, glucose, lactose, guar gum, hydroxypropyl cellulose, low-substituted hydroxypropyl cellulose, hydroxypropyl methylcellulose, hydroxypropyl methylcellulose acetate succinate, ethyl cellulose, methyl cellulose, ethyl cellulose, polyethylene oxide, sodium alginate, povidone, starch, pregelatinized starch, ammonia methacrylate copolymer and the like, or mixtures thereof.

According to present invention, diluent may include, but not limited to lactose anhydrous, lactose monohydrate, spray dried lactose, dicalcium phosphate, calcium phosphate tribasic, calcium carbonate, calcium sulfate, starch, corn starch, potato starch, wheat starch, pregelatinized starch, microcrystalline cellulose, silicified microcrystalline cellulose, cellulose microcrystalline powdered and the like, or mixtures thereof.

According to present invention, lubricant may include, but not limited to calcium stearate, glyceryl behenate, magnesium stearate, mineral oil light, polyethylene glycol, castor oil, sodium stearyl fumarate, starch, stearic acid, talc, paraffin, hydrogenated vegetable oil, zinc stearate, sodium lauryl sulphate, sodium benzoate, PEG 400,600 and the like, or mixtures thereof.

According to present invention, glidant may include, but not limited to calcium silicate, magnesium silicate, colloidal silicon dioxide, cornstarch, talc and the like, or mixtures thereof.

According to present invention disintegrants may include but not limited to traditional disintegrants, such as starch, and super disintegrants, which include polyvinylpyrrolidone, croscarmellose sodium, crospovidone, sodium starch glycolate, sodium carboxymethyl cellulose, calcium carboxymethyl cellulose, methyl cellulose, microcrystalline cellulose, powdered cellulose, lower alkyl-substituted hydroxypropyl cellulose, polacrilin potassium, starch, pregelatinized starch, sodium alginate, and mixtures thereof.

According to present invention, pH modifiers may include but not limited to acidic pH modifiers (e.g., acids such as citric acid, malic acid or succinic acid) that retard the dissolution of the pharmaceutical composition when the dispersion polymer is anionic. Alternatively, basic pH modifiers (e.g., sodium acetate or amines) enhance the rate of dissolution of the same types of pharmaceutical formulation.

According to present invention modified release matrix material may include, but not limited to hydroxypropyl methylcellulose (HPMC) with different viscosity grade; for example, HPMC K4M, HPMC K100M, HPMC K100LV, hydroxypropylcellulose, hydroxymethylcellulose, hydroxyethylcellulose, hydroxypropyl ethylcellulose, methylcellulose, ethylcellulose, carboxyethylcellulose, carboxymethyl hydroxyethylcellulose, carbomer, sodium carboxymethylcellulose, polyvinylpyrrolidone, hydrophobic polymers, waxes, fats, long-chained fatty acids, fatty alcohols or corresponding esters or ethers or their mixtures, and the like, or mixtures thereof.

According to present invention modified release coating material may include, but not limited to alkyl celluloses such as, e.g. ethyl cellulose, or cellulose esters, such as, e.g. cellulose acetate, cellulose acetate phthalate, hydroxylpropyl methyl cellulose phthalate, poly vinyl acetate phthalate, polymethacrylate, copolymer of ethyl acrylate and methyl methacrylate, ammonia methacrylate copolymers, polyacrylic acid and polyacrylate and methacrylate copolymers, shellac, hydroxyethyl cellulose, hydroxypropyl cellulose, hydroxypropyl methylcellulose, polyvinyl pyrrolidone, and the like, or mixtures thereof.

According to present invention the disintegration time of the immediate release formulation is an oral administration tablet is less than 15 minutes to 1 hour.

According to present invention the particle size distribution would be in the range of D90; 1 micron to 40 microns and 10 microns to 60 microns.

### EXAMPLES

The present invention has been described by way of example only, and it is to be recognized that modifications thereto falling within the scope and spirit of appended claims, and which would be obvious to a person skilled in the art based upon the disclosure herein, are also considered to be within the scope of this invention.

Immediate Release formulations for 5-flurorpyrimidine Derivative:
API details:

| | |
|---|---|
| IUPAC Name | (2R, 3R, 4R, 5R)-2-(5-Fluoro-4- |
| | octanamido 2-oxopyrimidine-1(2H)-yl)-5- |
| | methyl-tetrahydrofuran-3,4-diyl-diacetate |
| Chemical structure | 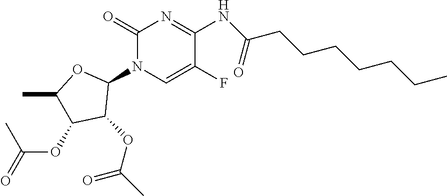 [SMILES: CCCCCCCC(=O)Nc1nc(=O)n([C@@H]2O[C@H](C)[C@@H](OC(C)=O)[C@H]2OC(C)=O)cc1F] |
| Reference code | CLX-155 |
| Molecular | C21H30FN3O7 |
| Formula Molecular weight | 455.21 (Diacetate Salt) |
| Solubility | Freely soluble in Methanol, Slightly Soluble in Acetonitrile |
| Wavelength Maxima | 249.0, 216 and 309.3 nm |
| Storage | Storage conditions: 2-8° C. |
| Melting point | 114-120° C. |

In certain embodiments, the immediate release formulation comprises of binders selected from non-limiting examples of PVP K 30 (BASF, Kollidon), Pregelatinised Starch (Colorcon), Hydroxy Propyl cellulose (Ashland, Klucel LF). Hypromellose thereof; Super disintegrants selected from non-limiting examples of sodium starch glycolate (Roquette, SSG Type-A), Croscarmellose sodium (FMC, Ac-Di-Sol SD 711); glidants selected from non-limiting examples Colloidal silicon dioxide (Evonik, Aerosil 200 Pharma), Talc (Imerys, Talc, Luzenac Pharma M). Surfactant selected from non-limiting examples of sodium lauryl sulfate (BASF, Kolliphor SLS), ammonium lauryl sulfate, dioctyl sodium sulfosuccinate, perfluorooctanesulfonate, Lauryldimethylamine oxide, Tween80, and bile salts; diluents selected from non-limiting examples lactose, microcrystalline cellulose, mannitol, dicalcium phosphate; and film coat examples is opadry clear YS-1-7006.

### Example 1: Formulation with Aqueous Wet Granulation Approach

Formulation 1 (With Microcrystalline cellulose and Hypromellose)

| Sr. No. | Ingredients | mg/Tablet | % w/w composition |
|---|---|---|---|
| 1 | API (CLX - 155) | 500.00 | 62.5 |
| 2 | Microcrystalline | 228.00 | 28.5 |
| 3 | Hypromellose | 40.00 | 5.0 |
| 4 | Purified water | q.s. | q.s. |
| 5 | Croscarmellose sodium | 24.00 | 3.0 |
| 6 | Magnesium stearate | 8.00 | 1.0 |

| | | |
|---|---|---|
| Uncoated Tablet weight (mg) | 800.00 | 100.00 |

### Manufacturing Procedure of Example 1:

1) Sift API CLX-155 and Microcrystalline cellulose through #40 mesh.
2) Load the blend of step No. 1 in Rapid mixer granulator (RMG) bowl followed by dry mixing for sufficient time.
3) Dissolve Hypromellose in suitable quantity of purified water.
4) Add slowly solution of step no. 3 onto step no. 2, to have uniform distribution of binder followed by granulation.
5) If required, wet mill the mass by using suitable screen followed by drying to achieve desired LOD.
6) Sift the dried granule by using suitable screen followed by blending with extragranular croscarmellose sodium in suitable blender.
7) Lubricate the blend of step No. 6, by using Magnesium stearate sifted through suitable screen.
8) Finally perform compression using suitable tooling's.

### Example 2: Formulation with Aqueous Wet Granulation Approach

Formulation 2 (With Microcrystalline
cellulose, Lactose and Povidone)

| Sr No. | Ingredients | mg/Tablet | % w/w composition |
|---|---|---|---|
| 1 | API (CLX - 155) | 500.00 | 62.5 |
| 2 | Microcrystalline | 100.00 | 12.5 |
| 3 | Lactose Monohydrate | 128.00 | 16.0 |
| 4 | Povidone | 40.00 | 5.0 |
| 5 | Purified water | q.s. | q.s. |
| 6 | Croscarmellose sodium | 24.00 | 3.0 |
| 7 | Magnesium stearate | 8.00 | 1.0 |

| | | |
|---|---|---|
| Uncoated Tablet weight (mg) | 800.00 | 100.00 |

### Manufacturing Procedure of Example 2:

1) Sift API CLX-155 and Microcrystalline cellulose and Lactose Monohydrate through #40 mesh.
2) Load the blend of step No. 1 in RMG bowl followed by dry mixing for sufficient time.
3) Dissolve Povidone in suitable quantity of purified water.
4) Add slowly solution of step no. 3 onto step no. 2, to have uniform distribution of binder followed by granulation.
5) If required, wet mill the mass by using suitable screen followed by drying to achieve desired LOD.
6) Sift the dried granule by using suitable screen followed by blending with extragranular croscarmellose sodium in suitable blender.
7) Lubricate the blend of step No. 6, by using Magnesium stearate sifted through suitable screen.
8) Finally perform compression using suitable tooling's.

### Example 3: Formulation with Non Aqueous Wet Granulation Approach

| Sr. No | Ingredients | mg/Tab |
|---|---|---|
| 1 | API (CLX -155) | 500 |
| 2 | Crospovidone (polyplasdone Ultra) | 20 |
| 3 | Sodium Lauryl Sulfate (kolliphor SLS fine) | 25 |
| 4 | Microcrystalline cellulose (Avicel PH112) | 99 |
| 5 | PVP K30 (Kollidon 30 LP) | 6 |
| 6 | Isopropyl alcohol | q.s |
| 7 | Crospovidone (polyplasdone Ultra) | 15 |
| 8 | Magnesium stearate | 5 |

| | |
|---|---|
| Uncoated Tablet weigh (mg) | 670 |

### Manufacturing Procedure of Example 3:

1. Binder solution preparation: Dissolved PVP K 30 (kollidon 30 LP) in isopropyl alcohol under stirring in a glass beaker till a clear solution was observed.
2. Mixed and co-sifted CLX-155, Sodium lauryl sulfate (kolliphor SLS fine), Crospovidone (polyplasdone ultra) and Microcrystalline cellulose (avicel PH 112) through 20 # was transferred into a RMG bowl.
3. Granulation—Binder solution and an additional quantity of isopropyl were added to complete the granulation.
4. Wet sifting: The granulated wet mass was sifted through 12 # screen.
5. Drying: Wet sifted granules were dried in fluidized bed dryer at 45-55° C. till the required LOD (NMT 1.50% w/w) was achieved.
6. Sizing: Sifted the dried granules through 20 # screen.
7. Prelubrication: Blended the granules with polyplasdone ultra
8. Lubrication—Granules were lubricated with magnesium stearate (60 # pass) in a cone blender
9. Compressed the lubricated blend by using 17.5×8 mm capsule shape tooling by using compression machine

### Example 3A: Formulation with Non Aqueous Wet Granulation Approach

| Sr. No. | Ingredients | mg/tab |
|---|---|---|
| 1 | API (CLX-155) | 500.00 |
| 2 | Crospovidone (Polyplasdone ultra) | 20.00 |
| 3 | Sodium lauryl sulfate (Kolliphor SLS fine) | 25.00 |
| 4 | Microcrystalline cellulose (Avicel PH 112) | 99.00 |
| 5 | PVP K 30 (Kollidon 30 LP) | 6.00 |
| 6 | Isopropyl alcohol | q.s. |
| 7 | Crospovidone (Polyplasdone ultra) | 15.00 |
| 8 | Magnesium stearate | 5.00 |

| | |
|---|---|
| Uncoated tablet weight (mg) | 670.00 |

### Details API:

| Lot No. | BCIEARW2002 |
|---|---|
| Assay | 99% |
| LOD (% w/w) | 0.32 |

### Manufacturing Procedure of Formulation 3A:

Note: Wet granulation was carried out in 2 sub lots and wet mass drying was done as a single lot.
1. Binder solution preparation: Dissolved PVP K 30 (kollidon 30 LP) in isopropyl alcohol under stirring in a glass beaker till a clear solution was observed
2. Mixed and co-sifted CLX-155, Sodium lauryl sulfate (kolliphor SLS fine), Crospovidone (polyplasdone ultra) and Microcrystalline cellulose (avicel PH 112) through 20 # was transferred into a RMG bowl.
3. Granulation-Binder solution and an additional quantity of isopropyl were added to complete the granulation.
4. Wet sifting: The granulated wet mass was sifted through 12 # screen.
5. Drying: Wet sifted granules were dried in fluidized bed dryer at 45-55° C. till the required LOD (NMT 1.50% w/w) was achieved.
6. Sizing: Sifted the dried granules through 20 # screen.
7. Prelubrication: Blended the granules with polyplasdone ultra
8. Lubrication-Granules were lubricated with magnesium stearate (60 # pass) in a cone blender
9. Compressed the lubricated blend by using 17.5×8 mm capsule shape tooling by using compression machine.

### Film Coating of Uncoated Tablets of Example 3A:

1. Uncoated tablets of example 3A were coated using non-aqueous coating dispersion of Opadry complete film coating system and color Red iron Oxide. The solvents used were isopropyl alcohol and dichloromethane.
2. The coating weight target was 2-3% w/w.

### Preparation Procedure for Coating Dispersion

1. Solvents were taken in a beaker
2. Opadry and color were added under stirring
3. Coating dispersion was strained through 250-micron muslin cloth
4. Uncoated tablets were loaded in the coating pan, prewarmed and film coated to the target weight
5. Tablets were stored in a securely closed airtight HDPE container along with silica gel pouch at 2-8° C.
6. Tablets were stored in a securely closed airtight HDPE container along with silica gel pouch at 2-8° C.
7. Coated tablets were packed and loaded on stability.

### Example 4: Formulation with Dry Granulation Approach

| Sr. No. | Ingredients | mg/Tab |
|---|---|---|
| 1 | API (CLX - 155) | 500 |
| 2 | Hydroxypropyl cellulose (Klucel ExF) | 12.50 |
| 3 | Crospovidone (Polyplasdone Ultra 10) | 12.50 |
| 4 | Sodium Lauryl sulfate (Kolliphor SLS Fine) | 20.00 |
| 5 | Microcrystalline cellulose (Avicel PH 101) | 105.00 |
| 6 | Crospovidone (Polyplasdone Ultra 10) | 15.00 |
| 7 | Magnesium stearate | 5.00 |

| | |
|---|---|
| Uncoated Tablet weight (mg) | 670.00 |

Dry granulation is also referred as pre-compression or double compression and is an agglomeration process designed to improve the flow and compression characteristics of powders that would otherwise have poor flow &/or compressibility. The process involves compaction of powder particles into larger pieces or compacts which are subsequently broken down into granules that can be further processed into dosage forms. Dry granulation is suitable for the compound which is sensitive to moisture and temperature. Since CLX-155 was found to be moisture sensitive and thermolabile, dry granulation strategy was explored evaluated.

### Manufacturing Procedure of Example 4:

i. Co-sifted CLX155, Hydroxypropyl cellulose (Klucel ExF), Crospovidone (Polyplasdone Ultra 10), Sodium Lauryl sulfate (Kolliphor SLS Fine) and Microcrystalline cellulose (Avicel PH 101) through 30 # and mixed in a polybag.
ii. Powder blend was slugged on compression machine.
iii. These slugs were crushed to obtain granules and passed through 20 #.
iv. The sifted granules were blended with Crospovidone and Magnesium stearate.
v. The lubricated blend was compressed by using 13 mm round, concave tooling.

### EQUIVALENTS

The present disclosure provides among other things pharmaceutically compositions and its methods of preparation for treating cancer and their complications. While specific embodiments of the subject disclosure have been discussed, the above specification is illustrative and not restrictive. Many variations of the systems and methods herein will become apparent to those skilled in the art upon review of this specification. The full scope of the claimed systems and methods should be determined by reference to the claims, along with their full scope of equivalents, and the specification, along with such variations.

### INCORPORATION BY REFERENCE

All publications and patents mentioned herein, including those items listed above, are hereby incorporated by reference in their entirety as if each individual publication or patent was specifically and individually indicated to be incorporated by reference. In case of conflict, the present application, including any definitions herein, will control.

We claim:

1. A pharmaceutically acceptable composition comprising:
a. an active ingredient selected from 5-fluropyrimidine derivatives, their isomers, stereoisomers, diastereomers, enantiomers, prodrug and pharmaceutically acceptable salts thereof in an amount of 100-3000 mg and comprising at least 60-85% by weight of the formulation;
b. one or more disintegrants or super disintegrants in an amount of 0.5 to 6% by weight;
c. one or more surfactants in an amount of 0.1 to 5% by weight;
d. one or more binders in an amount of 0.2 to 5% by weight; and
e. at least one lubricant in an amount of 0.3 to 3% by weight; wherein the pharmaceutically acceptable composition exhibits a friability of not more than 1 to 10%.

2. The pharmaceutically acceptable composition as claimed in claim 1, wherein the composition comprises at least 500 mg 5-fluropyrimidine; derivative wherein 5-fluropyrimidine is derivative is (2R, 3R, 4R, 5R)-2-(5-Fluoro-4-octanamido2-oxopyrimidine-1 (2H)-yl)-5-methyl-tetrahydrofuran-3,4-diyl-diacetate.

3. The pharmaceutically acceptable composition as claimed in claim 1, wherein the one or more disintegrants or super disintegrants is selected from polyvinylpyrrolidone, croscarmellose sodium, crospovidone, sodium starch glycolate, sodium carboxymethyl cellulose, calcium carboxymethyl cellulose, methyl cellulose, microcrystalline cellulose, powdered cellulose, lower alkyl-substituted hydroxypropyl cellulose, polacrilin potassium, starch, pregelatinized starch and sodium alginate.

4. The pharmaceutically acceptable composition as claimed in claim 1, wherein the one or more binders is selected from acacia, carbomer, carboxymethylcellulose, cellulose microcrystalline, copovidone, gelatin, glucose, lactose, guar gum, hydroxypropyl cellulose, low-substituted hydroxypropyl cellulose, hydroxypropyl methylcellulose, hydroxypropyl methylcellulose, ethyl cellulose, acetate succinate, methyl cellulose, ethyl cellulose, polyethylene oxide, sodium alginate, povidone, starch, pregelatinized starch, ammonia methacrylate copolymer.

5. The pharmaceutically acceptable composition as claimed in claim 1, wherein the one or more surfactant is selected from sodium lauryl sulfate, ammonium lauryl sulfate, dioctyl sodium sulfosuccinate, perfluorooctanesulfonate, lauryldimethylamine oxide, Tween80, bile salts and thereof.

6. The pharmaceutically acceptable composition as claimed in claim 1, wherein the at least one lubricant is selected from calcium stearate, glyceryl behenate, magnesium stearate, mineral oil light, polyethylene glycol, castor oil, sodium stearyl fumarate, starch, stearic acid, talc, paraffin, hydrogenated vegetable oil, zinc stearate, sodium lauryl sulphate, sodium benzoate, PEG 400 and PEG 600.

7. The pharmaceutically acceptable composition as claimed in claim 1, wherein the composition is an immediate release, modified release composition such as sustained release, prolonged release, delayed release, pulsed release, controlled release, accelerated release, fast release, targeted release, gastric retention dosage release and thereof.

8. The pharmaceutically acceptable composition as claimed in claim 1 wherein the composition is administered for treating cancer.
